(19) United States
(12) Patent Application Publication
(10) Pub. No.: US 2024/0415696 A1
(43) Pub. Date: Dec. 19, 2024

(54) ROBOTIC OCULAR NEEDLE DELIVERY DEVICE

(71) Applicant: Alcon Inc., Fribourg (CH)

(72) Inventors: Patrick TERRY, Goleta, CA (US); Satish YALAMANCHILI, Lake Forest, CA (US); Russell FINLAY, Fort Worth, TX (US); Nanhong LOU, Fort Worth, TX (US)

(21) Appl. No.: 18/677,618

(22) Filed: May 29, 2024

Related U.S. Application Data
(60) Provisional application No. 63/507,990, filed on Jun. 13, 2023.

(51) Int. Cl. A61F 9/00; A61M 5/158; G06T 7/00

(52) U.S. Cl. CPC A61F 9/0017; G06T 7/0012; A61M 2005/1585; A61M 2210/0612; G06T 2207/30041

(57) ABSTRACT

A delivery device includes a conveyor, one or more imaging devices configured to have an eye of a patient in a field of view thereof, and an needle assembly configured to receive a drug to be injected into the eye of the patient. A staging assembly is mounted to the conveyor and includes one or more actuators configured to position the needle assembly relative to the eye of the patient. A controller is configured to receive one or more images from the one or more imaging devices; detect a location of anatomy of the eye of the patient in the one or more images; and activate the one or more actuators to drive a needle mounted to the needle assembly into a placement location on the eye of the patient according to the location of the anatomy. The controller activates the needle assembly to inject fluid into the eye or draw tissue out of the eye.

### CROSS-REFERENCE TO RELATED APPLICATIONS

This application claims benefit of and priority to U.S. Provisional Patent Application No. 63/507,990, filed Jun. 13, 2023, which is hereby assigned to the assignee hereof and hereby expressly incorporated by reference in its entirety as if fully set forth below and for all applicable purposes.

### INTRODUCTION

The present disclosure relates generally to instruments used for providing intravitreal injections and other types of injections of the eye or the orbital space of the eye, such as intracameral injections, subretinal injections, suprachoroidal injections, subconjunctival injections, retro-orbital injections, periorbital injections, and the like. The present disclosure also relates to the extraction of tissue, such as for performing a biopsy.

Light received by the eye is focused by the cornea and lens of the eye onto the retina at the back of the eye, which includes the light sensitive cells. The interior of the eye between the lens and the retina is filled with a transparent gel known as the vitreous. Many conditions of the retina are treated by intravitreal injections in which medication is injected into the vitreous. Such conditions include age-related macular degeneration, retinal vein occlusion, diabetic macular edema, diabetic retinopathy, and others. Once diagnosed with a condition treated by intravitreal injections, a patient may continue to receive injections periodically. Other conditions may be diagnosed by a biopsy, which likewise requires the precise insertion of a needle.

It would therefore be an advancement in the art to facilitate the administration of ocular and orbital injections and perform ocular and orbital biopsies.

### SUMMARY

In certain embodiments, a delivery device includes a conveyor, one or more imaging devices configured to have an eye of a patient in a field of view thereof, and an needle assembly including a needle. A staging assembly is mounted to the conveyor and includes one or more actuators. The conveyor is configured to move the staging assembly in three-dimensional space. The staging assembly is configured to position the needle assembly relative to the eye of the patient. A controller is coupled to the conveyor, the one or more imaging devices, and the staging assembly. The controller is configured to receive one or more images from the one or more imaging devices; detect a location of anatomy of the eye of the patient in the one or more images; and activate the one or more actuators to drive a needle mounted to the needle assembly into a placement location on the eye of the patient according to the location of the anatomy.

In certain embodiments, a method for drug delivery includes activating, by a controller, a conveyor to transport a staging assembly toward an eye of a patient, the staging assembly having an needle assembly mounted thereto; receiving, by the controller, one or more images from one or more imaging devices mounted to the staging assembly; detecting a location of anatomy of an eye of the patient in the one or more images; activating, by the controller, one or more actuators of the staging assembly to align the needle assembly relative to the location of the anatomy; and activating, by the controller, the one or more actuators to drive a needle mounted to the needle assembly into a placement location on the eye of the patient according to the location of the anatomy.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a side view of a robotic needle delivery device in accordance with certain embodiments.

FIG. 2A is a top view of a docking assembly of the robotic needle delivery device in accordance with certain embodiments.

FIG. 2B is a partial side view of the device of FIG. 2A.

FIG. 2C is a top view of a docking assembly including a speculum in accordance with certain embodiments.

FIG. 3 is a side view of an alternative robotic needle delivery device in accordance with certain embodiments.

FIG. 4 is a top view of a staging assembly for the alternative robotic needle delivery device in accordance with certain embodiments.

FIGS. 5A to 5D illustrate example needle assemblies in accordance with certain embodiments.

FIG. 6 is a schematic block diagram of electronic components of a multi-station robotic needle delivery device in accordance with certain embodiments.

FIG. 7 is a process flow diagram of a method for preparing for needle insertion using a robotic needle delivery device in accordance with certain embodiments.

FIG. 8 is a process flow diagram of a method for needle insertion using a robotic needle delivery device in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

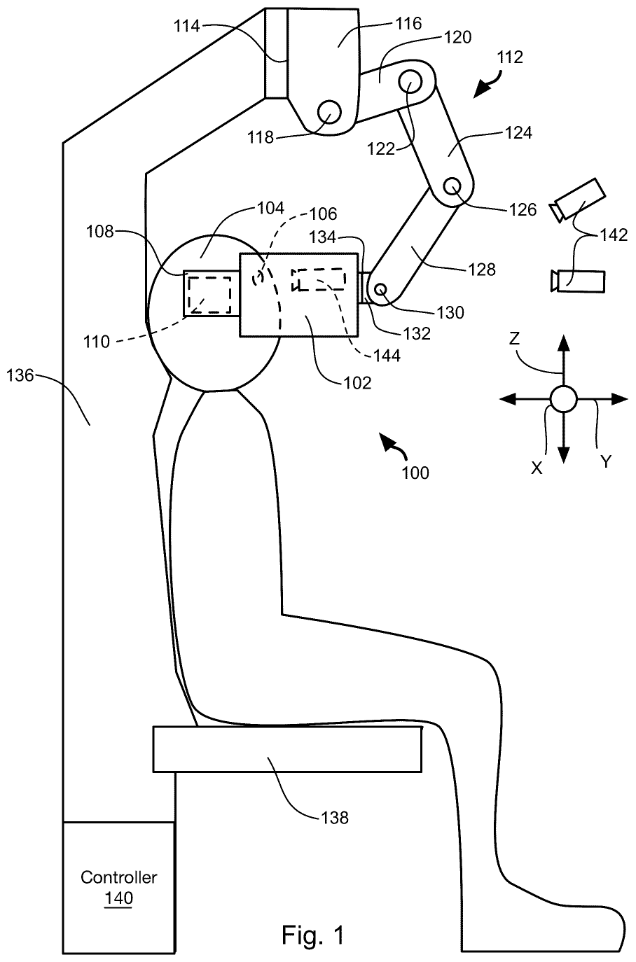

FIG. 1 illustrates an example embodiment of an example multi-station robotic needle delivery device 100 (hereinafter “needle delivery device 100”). The needle delivery device 100 includes a docking assembly 102 that receives a portion of the head 104 of a patient and covers one or both eyes 106 of the patient. The docking assembly 102 may include some or all of a forehead rest, cheek rests, temporal rests, chin rest, or rests for engaging other portions of the head 104 of the patient, some or all of which may be adjustable. The docking assembly 102 may include structures for clamping or otherwise retaining the head 104 of the patient, such as one or more clamping actuators 108 for pressing pads 110 against the patient's head 104, a headband encircling the patient's head 104 and fastened to the docking assembly 102, or other structures. Alternatively, the patient may be relied upon to press the patient's head against the docking assembly 102 with sufficient stability for the administration of an injection as described below.

The docking assembly 102 includes a conveyor 112 for moving the docking assembly 102 into alignment with the head 104 of patients of various sizes. The conveyor 112 may be understood with respect to X, Y, and Z direction, where the Z direction is substantially (e.g., within 2 degrees of) parallel to the direction of gravity and the X and Y directions are substantially (e.g., within 2 degrees of) perpendicular to the Z direction and to one another. The conveyor 112 is configured to move the docking assembly 102 in the X, Y, and Z directions as well as one or more rotational degrees of freedom, such as rotation about an axis substantially (e.g., within 2 degrees of) parallel to the X, Y, and/or Z direction.

The conveyor 112 may be embodied as robotic arm including a rotational joint 114 coupled to a base 116. The base 116 is coupled by elbow joint 118 to a link 120. Elbow joint 122 couples link 120 to link 124. Elbow joint 126 couples link 124 to link 128. Elbow joint 130 couples link 128 to link 132. Link 132 may be coupled by rotational joint 134 to the docking assembly 102. Each of the illustrated joints 114, 118, 122, 126, 130, 134 has a corresponding actuator for inducing movement of the joint. The conveyor 112 may have at least five degrees of freedom. For example, the illustrated conveyor 112 has six degrees of freedom (DOF). The conveyor 112 may be embodied as a commercially available serial robotic arm. The conveyor 112 may also be implemented as linear actuators, such as linear actuators implementing movements in the X, Y, and Z directions as well as one or more rotational actuators inducing rotation about one or more of the X, Y, and Z axes. For example, the conveyor 112 may be embodied as gantry. Note that the illustrated size of the conveyor 112 may be somewhat exaggerated relative to the size of the patient's head 104 and may have a smaller relative size. For example, the conveyor 112 may move the docking assembly within a three-dimensional range of motion having dimensions in the X, Y, and Z directions that are less than 30 centimeters, 15 centimeters, or 10 centimeters.

The conveyor 112 may be mounted to a support 136, such as by the rotational joint 114 mounting the illustrated robotic arm to the support 136. The support 136 may be mounted to a floor, wall, ceiling, movable cart, or other structure. A seat 138 on which a patient sits when the patient's head 104 is in the docking assembly 102 may be mounted to the support 136 or secure to a floor or wall adjacent the support 136. A harness may secure the patient to the seat 138 and/or support 136 to reduce movement of the head 104 of the patient relative to the docking assembly 102. The docking assembly 102, support 136, or other structure, may have speakers mounted thereto to which a medical professional can wirelessly connect and output verbal instructions or reassurance to the patient.

The docking assembly 102 and actuators of the conveyor 112 may be coupled to a controller 140. The controller 140 may be housed within the support 136 or elsewhere. The controller 140 may receive images from one or more cameras 142 in order to estimate a three-dimensional position of the patient's head 104 and activate the conveyor 112 to position the docking assembly 102 at or within a threshold distance of the patient's head 104. The docking assembly 102 itself may include one or more cameras 144. Images from the one or more cameras 144 may be used by the controller 140 to perform fine adjustments to the position of the docking assembly 102. Alternatively, the docking assembly 102 may incorporate actuators that are controlled to perform fine adjustments of the docking assembly 102 based on one or more images from the one or more cameras 144.

The position of the docking assembly 102 itself may be determined by sensing a kinematic state of the conveyor 112 using sensors incorporated into the joints 114, 118, 122, 126, 130, 134 or elsewhere in the conveyor 112. Alternatively or additionally, the position of the docking assembly 102 may also be determined based on images from the one or more cameras 142, 144.

Although cameras 142, 144 are described as being used to estimate the position of the patient's head 104 and possibly the docking assembly 102, other imaging or sensing modalities may be used such as light detection and ranging (LIDAR), radio detection and ranging (RADAR), ultrasonic sensing, or other type of sensor. The one or more cameras 144 may each be replaced with an optical coherence tomography (OCT) device, scanning laser ophthalmoscope, or other type of imaging device. An OCT device is particularly helpful for tracking the location of a needle during insertion, injection, and withdrawal.

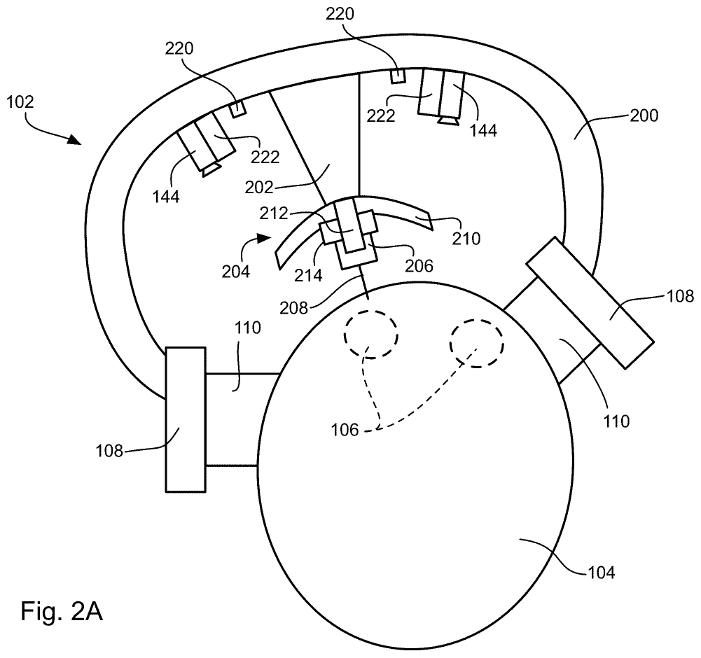

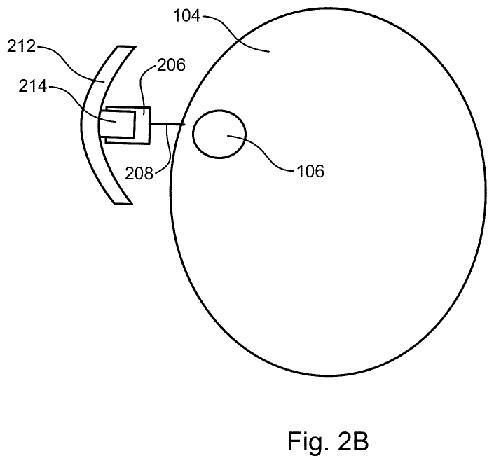

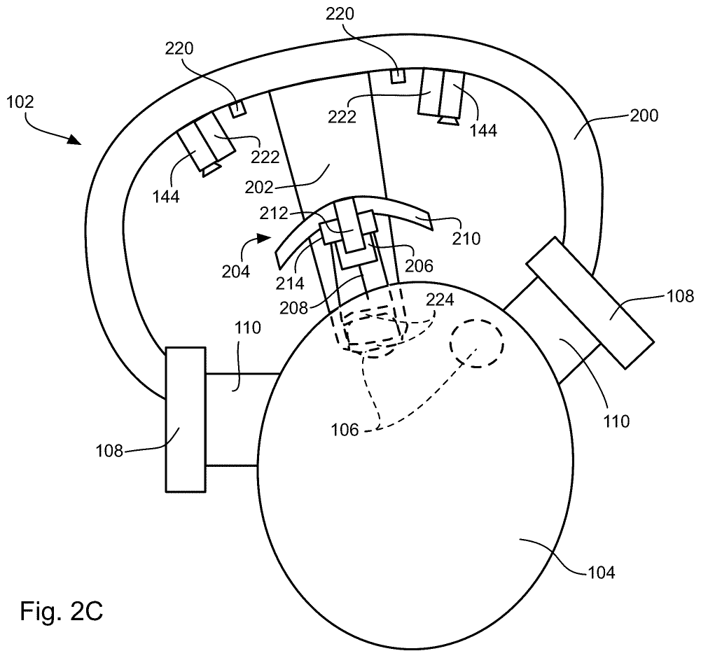

Referring to FIGS. 2A and 2B, the docking assembly 102 may include a frame 200. The frame 200 may be embodied as a track, rail, or other structural member along which components may be fastened at various positions. The frame 200 may include one or more mounting structures 202 mounted thereto. Each mounting structure 202 has a staging assembly 204 mounted thereto. The staging assembly 204 includes one or more actuators that perform fine adjustments, relative to the precision of the conveyor 112, of the position of the needle assembly 206 for inserting a needle 208 of the needle assembly 206 into the eye 106 of the patient in order to inject a drug or draw out tissue, such as for a biopsy.

In the illustrated embodiment, the staging assembly 204 includes an actuator 210 and an actuator 212 that are oriented substantially (e.g., within 2 degrees of) perpendicular to one another. The actuators 210, 212 may be linear actuators or the illustrated arcuate actuators 210, 212. For example, the actuators 210, 212 may define arcuate actuation paths that are each centered on a remote center of motion. For example, the remote center of motion may lie on the needle 208 or a path followed by the needle 208 when extended by an extension actuator 214, which is a linear actuator configured to extend and withdraw the needle 208 when performing injections. For example, the actuator 210 may be mounted to the mounting structure 202, the actuator 212 may be mounted to the actuator 210 and be actuated thereby along a first actuate path. The extension actuator 214 may be mounted to the actuator and may be actuated thereby along a second arcuate path that has the same remote center of motion as the first arcuate path, e.g., within 1 mm, 0.01 mm, or 1 micron. The needle assembly 206 may be mounted to the extension actuator 214 with the needle 208, or a line extending along the center of the lumen of the needle 208 lying on the remote center of motion, e.g., within 1 mm, 0.01 mm, or 1 micron.

The one or more clamping actuators 108 may be mounted to the frame 200. The clamping actuators 108 are configured to extend one or more pads 110 into engagement with the head 104 of the patient in order to reduce movement of the head of the patient relative to the docking assembly 102. For example, there may be two pads 110 with one pad or both pads being coupled to clamping actuators 108 for decreasing the distance between the two pads 110 in order to clamp the head 104 of the patient.

In practice, the docking assembly 102 is positioned relative to the head 104 of the patient using the conveyor 112 and images from the one or more cameras 142. One or more images from the one or more cameras 144 of the docking assembly 102 may be used to determine the relative position of the eye 106 of the patient and perform fine adjustments using the conveyor 112 based on the position. Once in position, the clamping actuators 108 may be activated to bring the pads 110 into engagement with the head 104 of the patient. Note that the position of the pads 110 may be asymmetric relative to the head 104 of the patient since the same docking assembly 102 may be used in two different positions to perform injections on the right and left eyes 106 of the patient. The actuation of the clamping actuators 108 may be guided by images from the one or more cameras 144. For example, the clamping actuators 108 may be used to adjust the relative positions of the docking assembly 102 and the patient's head 104.

The goal of positioning of the docking assembly 102 may be to position the needle 208 on a line that intersects a point on the eye 106 of the patient at a prescribed position and angle, or within a tolerance of such a position and angle that is within the range of motion provided by the staging assembly 204. For example, when performing intravitreal injection, the prescribed position may be between 3 and 3.5 millimeters from the limbus for an aphakic eye and between 3.5 and 4 millimeters from the limbus for a phakic eye. The prescribed angle may be determined as known in the art of intravitreal injections and may be selected such that upon insertion of the needle, the needle avoids contact with the lens and retina while placing medication near the retina or area of the retina to be treated.

The docking assembly 102 may include one or more electronic components in addition to the one or more cameras 144. The docking assembly 102 may include one or more fixation targets 220. Each fixation target 220 may be embodied as a static image, light source, screen for displaying a fixation target, or other device. A separate fixation target 220 may be provided for each eye 106 or a single fixation target 220 may be used for both right and left eyes 106. Alternatively, a single fixation target 220 may be mounted at different positions on the frame 200 for different eyes 106. In some embodiments a single fixation target 220 is centrally located to be used for both eyes 106, i.e., patient may direct each eye 106 toward the nose of the patient in order to expose the sclera for receiving an injection. Alternatively, a single screen implementing the fixation target 220 may display a fixation target at a different location for each eye 106. The location of the fixation target 220 may be adjusted using software executed by the controller 110 or by an observer in order to induce the patient to position the eye 106 at a desired angle

The docking assembly 102 may include one or more intraocular pressure (IOP) sensors 222. The IOP sensor 222 may be a contact or non-contact sensor and may be used during intravitreal injection to ensure that the TOP of the patient's eye 106 does not increase to unsafe levels. There may be separate IOP sensors 222 for each eye or a single IOP sensor 222 may be mounted at different positions on the frame 200 in order to measure the IOP of each eye 106.

Referring to FIG. 2C, in some embodiments, the docking assembly 102 may additionally include a speculum 224. The speculum 224 engages the eyelid of the patient and withdraws the eyelid to reduce risk of interference with injection of the needle 208. The speculum 224 may incorporate a force/torque sensor coupled to the controller 140, which uses the output of the force/torque sensor to limit the amount of pressure applied to the patient's eye 106 and eyelid. The speculum 224 may be statically mounted and engage the eyelid of the patient as the docking assembly 102 is brought into alignment with the eye 106 of the patient. Alternatively, the speculum 224 may include an extending actuator that extends the speculum 224 following alignment using one or both of the conveyor 112 and the staging assembly 204.

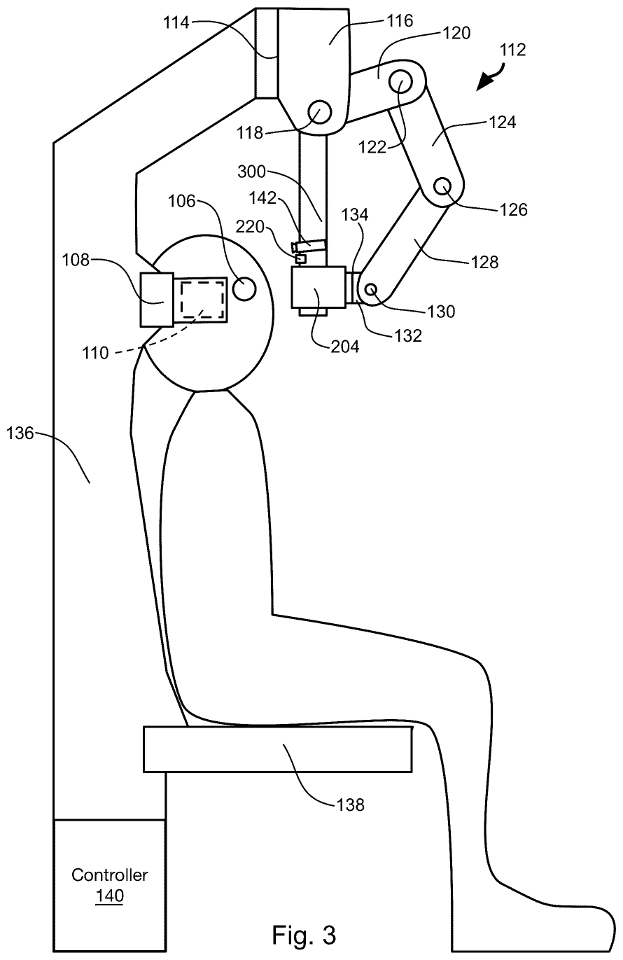

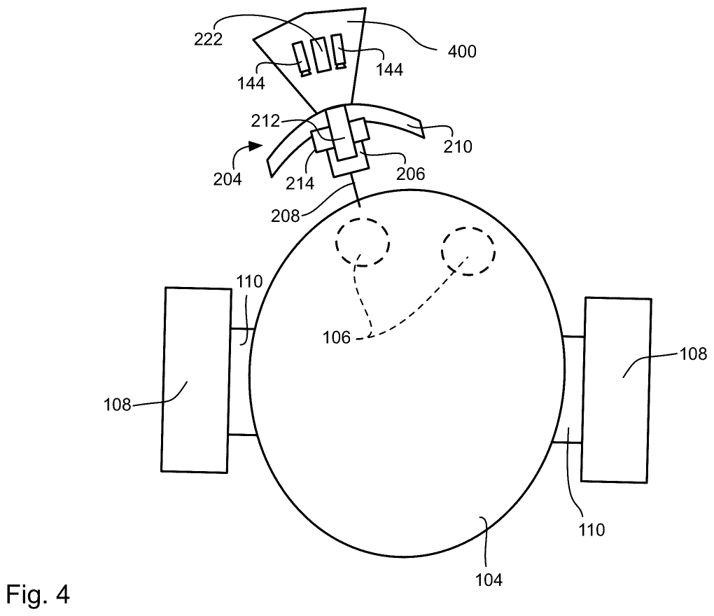

Referring to FIGS. 3 and 4, in an alternative implementation, one or two clamping actuators 108 mount to the support 136 and clamp the patient's head 104 between pads 110 in order to reduce movement of the patient's head 104 relative to the support. The clamping actuators 108 may be eliminated in some embodiments in favor of a passive or manually actuated clamp for clamping the patient's head 104. In the illustrated embodiments, components such as one or more cameras 142 and the fixation target 220 may mount to a support 300 that is fixed relative to the support 136. The staging assembly 204 may be mounted to the conveyor 112, which is then activated by the controller 140 to bring the staging assembly 204 into alignment with the patient's eye 106 and perform fine adjustments using the staging assembly 204 as described above.

The staging assembly 204 may mount to the conveyor 112 by means of a mounting structure 400. For example, the mounting structure 400 may mount to the joint 118. Some or all of the one or more cameras 144 and one or more IP sensors 222 may mount in a fixed relationship to the staging assembly 204, such as to the staging assembly 204 itself, to the mounting structure 400, or to some other structure secured to the mounting structure 400.

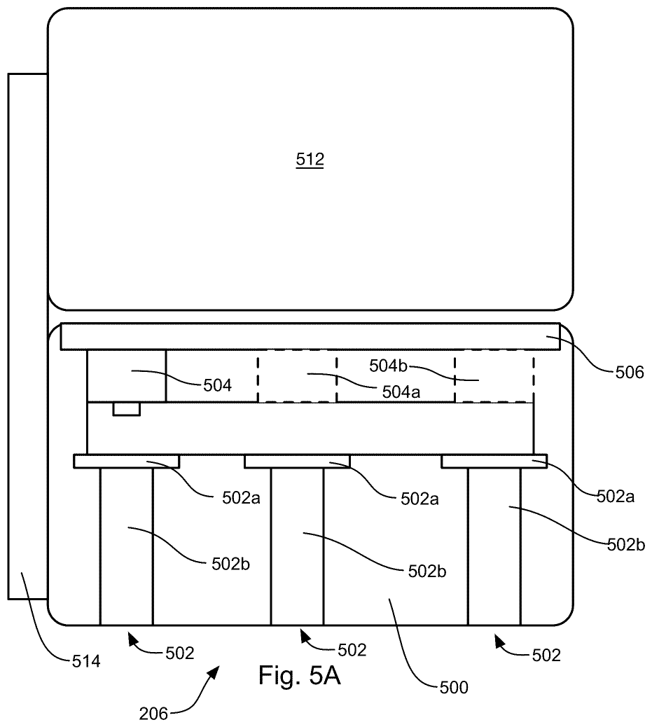

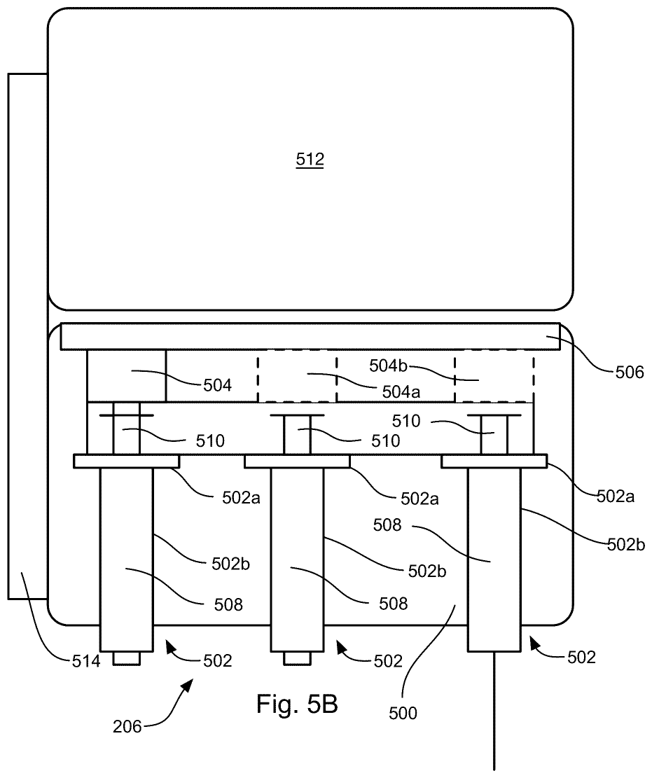

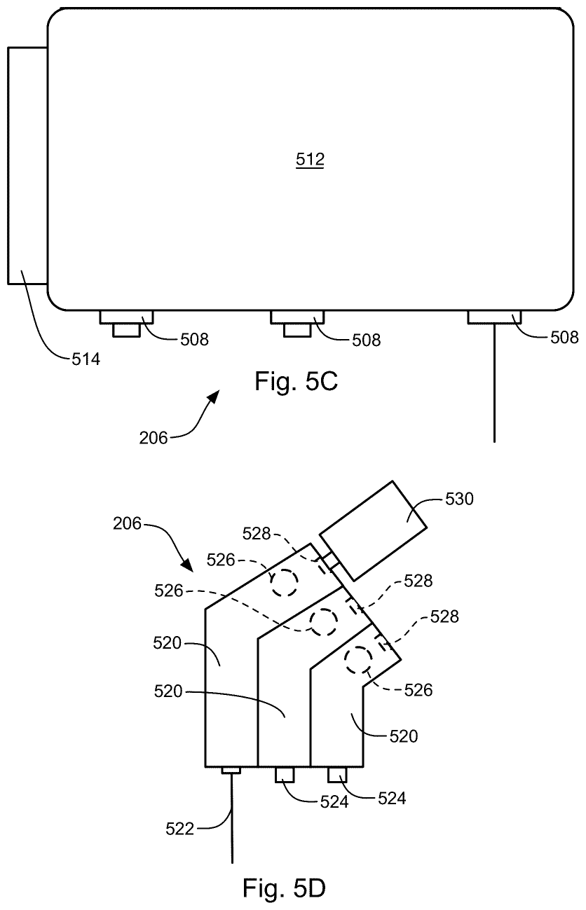

Referring to FIGS. 5A to 5C, the needle assembly 206 used for drug injection may have some or all of the attributes and/or functionalities described below. A needle assembly 206 used for drawing out tissue for a biopsy may include a pump controlled by the controller 140 for creating a vacuum within a needle for drawing out tissue. In either case, the supply of pressure or vacuum may be generated by a component of the needle assembly 206 or by an external source of pressurized air or vacuum.

The needle assembly 206 used for administering injections may include a tray 500 defining one or more recesses 502 for receiving syringes, such as three recesses 502 for receiving syringes containing an anesthetic, a disinfectant, and a drug to be delivered by injection. For example, each recess 502 may include a groove 502a for receiving a flange of a syringe and a recess 502b connected to the groove 502a for receiving the barrel of the syringe.

A plunger actuator 504 is positioned to depress the plunger 510 of syringes 508 positioned within the recesses 502. In some embodiment, a single plunger actuator 504 is used and is moved by a positioning actuator 506 between the illustrated position and two other positions 504a, 504b in order to depress the plunger 510 of syringes positioned in each of the recesses 502. In other embodiments, a separate plunger actuator 504 is provided to depress the plunger 510 of a syringe 508 positioned in each recess 502.

Syringes 508 may be retained within the recesses 502 by means of a lid 512 or other retention structure. The lid 512 may be coupled to a lid actuator 514 that can be moved into the open position of FIGS. 5A and 5B and into the closed position of FIG. 5C in which the lid 512 is positioned over the syringes 508 positioned within the recesses 502. The lid 512 may be flat or may include recesses that receive portions of the syringes 508 when the lid 512 is in the closed position over the tray 500. For example, the lid 512 may include recesses 502 similarly to the tray 500, each recess 502 including a groove 502a for receiving a flange of a syringe 508 and a recess 502b for receiving the barrel of a syringe 508.

FIG. 5D illustrates an alternative implementation for the needle assembly 206. In the illustrated embodiment, the needle assembly 206 includes one or more reservoirs 520, such as the illustrated reservoirs 520 for containing a drug to be administered, an anesthetic, and a disinfectant. The reservoirs 520 may be separate members or joined together by fasteners, placement in a common housing, or co-molding. Each reservoir 520 may have an outlet formed thereon or secured thereto, such as in the form of a hypodermic needle 522 or nozzle 524 for dispensing fluid.

Each reservoir 520 may have a pump 526 associated therewith. The pump 526 of each reservoir 520 may be used to force fluid out of the outlet of the reservoir 520. The pump 526 may be replaced with other propulsion sources. For example, pressurized fluid may be forced into a reservoir 520 and engage a piston or bladder in order to force fluid out of the reservoir 520.

Each reservoir 520 may have an inlet 528 for filling the reservoir 520. The inlet 528 may be coupled to a vial 530 or syringe containing fluid to be loaded into the reservoir 520. The fluid may be forced into the reservoir 520 using a syringe or other pressure source. Alternatively, the pump 526 of a reservoir 520 may be activated in order to draw fluid out of a vial 530 through the inlet 528 of the reservoir 520. In other implementations, fluid may be drawn through the inlet 528 or outlet of a reservoir 520 and into a bladder within a reservoir 520 by reducing pressure in the reservoir 520 around the bladder, such as through a port for coupling to a pneumatic pressure source. In some embodiments, the reservoirs 520 may be large enough to store multiple doses. In such embodiments, the needle assembly 206 may include refrigeration to avoid degradation of a drug to be injected. In some embodiments, the reservoirs 520 may be large enough to store multiple doses. In such embodiments, the needle assembly 206 may include refrigeration to avoid degradation of a drug to be injected.

The inlet 528 may be include a one-way valve, self-sealing polymer defining a hole for receiving a needle, removable cap, or other closure mechanism. In some embodiments, the needle assembly 206 is a disposable cartridge that is pre-loaded with fluid such that an inlet 528 is omitted. For example, the reservoirs 520 may be filled through the outlet thereof at the time of manufacture.

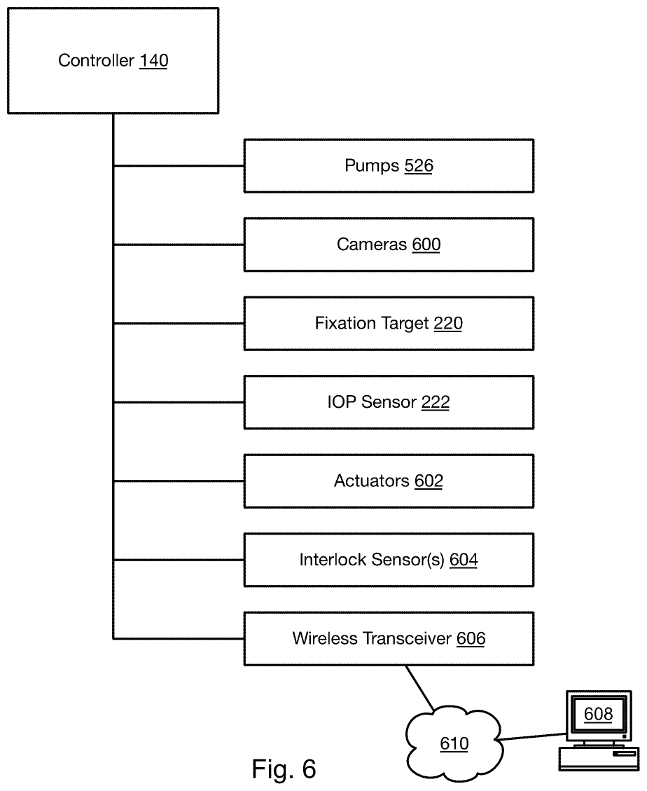

Referring to FIG. 6, the controller 140 is coupled to the one or more pumps 526, one or more cameras 600 (e.g., cameras 142, 144), the one or more IOP sensors 222, and the one or more fixation targets 220 by means of wired or wireless connections, optical fibers, or other type of connection. The controller 140 is configured to activate and deactivate the one or more pumps 526, one or more cameras 600, and one or more IOP sensors 222, and one or more fixation targets 220. The controller 140 is configured to receive images from the one or more cameras 600 and IOP readings from the one or more IOP sensors 222. The controller 140 may receive feedback from the one or more pumps 526, such as measurements of pressure at the input and/or output of each pump of the one or more pumps 526, current drawn by each pump of the one or more pumps 526, or other information. In some embodiments, feedback from the one or more pumps 526 may be used to obtain an estimated IOP reading and the one or more IOP sensors 222 may be omitted.

The controller 140 may be coupled to one or more other components, such as actuators 602 including some or all of the actuators of the conveyor 112, staging assembly 204, and needle assembly 206 described herein in order to control activation of the actuators 602 and possibly receive feedback regarding the state of each actuator 602 (e.g., current angular or translational position, velocity, and/or acceleration).

The staging assembly 204 may include electrical contacts coupled to the controller and which contact corresponding contacts on the needle assembly 206 in order to supply power and control signals to actuators 504, 504 or pumps 526 of the needle assembly 206 from the controller 140.

The controller 140 may be coupled to one or more interlock sensors 604 that detect a state of the needle delivery device 100 relative to the head 104 of the patient. For example, interlock sensors 604 may sense whether a patient's head 104 is clamped between the pads 110, whether the needle assembly 206 is properly mounted to the staging assembly 204, or that any of the components described herein is positioned and functioning properly.

The controller 140 may be coupled to a wireless transceiver 606. The operation of the controller 140 may be subject to authorization and instructions received from a computing device 608 over a network 610 by way of the wireless transceiver 606. The controller 140 may authenticate a user of the computing device 608 prior to permitting control using the computing device 608. In some embodiments, the needle delivery device 100 is used in a clinic or hospital in which medical supervision may be provided in-person or by a locally connected interface such that the wireless transceiver 606 may be omitted.

In some embodiments, the observer is remote and may interact with the patient during a procedure, such as by means of an output device such as a screen, speakers, or other device incorporated into the docking assembly 102. Instructions to the patient may be output from the output device either automatically or in response to instructions from the remote observer. The patient may interact with the remote observer using an input device incorporated into the drug docking assembly 102, such as the one or more cameras 142, a microphone, a touch screen, pointing device, a keyboard, or other input device.

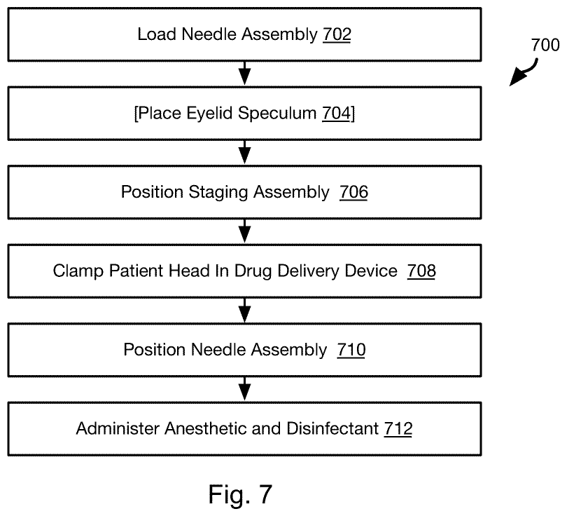

FIG. 7 is a process flow diagram of a method 700 for preparing for needle delivery using the needle delivery device 100. Where a drug is to be injected, the method 700 includes loading, at step 702, fluid into the needle assembly 206, including the drug to be delivered, an anesthetic, and a disinfectant as described above with respect to FIGS. 5A to 5D. As an alternative, a disposable needle assembly 206 may be provided that is already loaded with fluid such that step 702 is not performed by the patient. Step 702 may include locking the needle assembly 206 into place on the staging assembly 204. Where a biopsy is performed, step 702 may be omitted.

In some embodiments, the patient may place, at step 704, a speculum in the eye 106 to be treated in order to move the eyelid out of the way. In other embodiments, the patient is relied upon to maintain the eyelid out of the way such that a speculum is not used. In still other embodiments, an actuated speculum is incorporated into the staging assembly 204 (see FIG. 2C) and withdraws the eyelid automatically.

The method 700 may include positioning, at step 706, the staging assembly 204 in alignment with the patient's head 104 and the eye 106 to be treated. The positioning of step 706 may be performed by the conveyor 112 with guidance provided by images from the one or more cameras 142 and possibly the one or more cameras 144. The alignment of step 706 may be a rough alignment, such as alignment within a tolerance that is less than or equal to a range of motion of the staging assembly 204, such as at less than or equal to half the range of motion of the staging assembly 204 along the X, Y, and Z directions.

The method 700 includes clamping, at step 708, the patient's head 104 in the drug delivery device, such as by activating one or more actuators 108 to bring pads 110 into engagement with the patient's head 104. As noted above, the actuators 108 may be mounted to the docking assembly 102 or to the support 136.

The method 700 includes positioning, at step 710, the needle assembly 206. Step 710 may be performed using the arcuate actuators 210, 212 of the staging assembly 204 with guidance from images of the one or more cameras 144. The positioning of step 710 may include identifying a limbus of the patient's eye 106 in the images and positioning and orientating the needle 208 of the needle assembly 206 such that upon actuation of the extension actuator 214, the needle 208 will enter the patient's eye 106 at a prescribed point relative to the limbus and at a prescribed angle for performing an intravitreal injection. Other types of injections may be placed for insertion at different points on the eye 106 and may the needle 208 may be positioned by identifying the location of different anatomy of the eye.

The method 700 may include administering, at step 712, an anesthetic and a disinfectant. Step 712 may be an automated step in which each of the anesthetic and disinfectant is dispensed by depressing a plunger of a syringe using a plunger actuator 504 or activating a pump 526. The outlets of the syringes 508 or reservoirs 520 used to dispense the anesthetic and disinfectant may be placed close to the eye 106 being treated, e.g., within 1 millimeter, or in contact with the eye 106. Alternatively, fluid may be sprayed at step 712 such that such proximity is not required. In some embodiments, step 712 is performed manually by a patient prior to performing step 710.

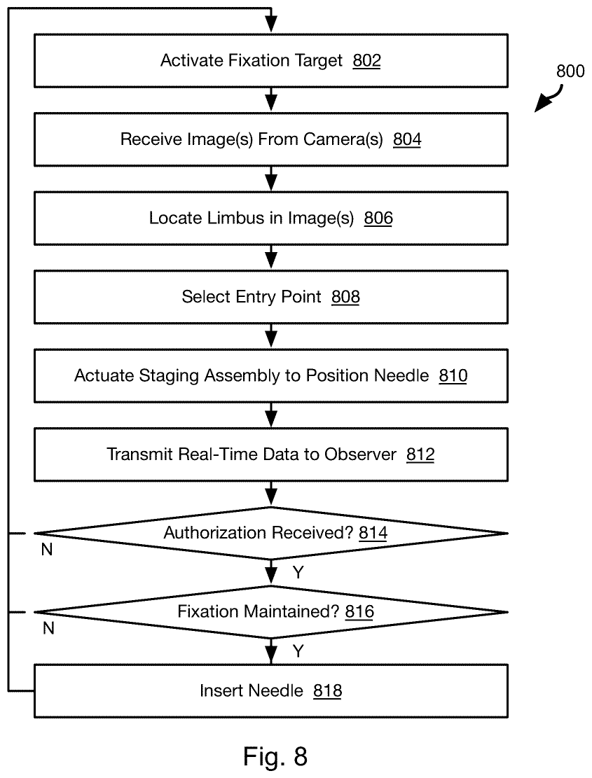

FIG. 8 is a process flow diagram of a method 800 for administering an injection or performing a biopsy using the needle delivery device 100. The method 800 may be performed following performance of the method 700 by the controller 140 activating components of the needle delivery device 102.

The method 800 includes activating, at step 802, a fixation target 220. Activating the fixation target 220 may include activating a light, e.g., light emitting diode, displaying an image on the screen, or otherwise providing a visual indicator that is visible to the eye 106 to be treated. Where the fixation target is a static visible structure, step 802 may be omitted. Step 802 may include outputting visual or audible instructions to the patient to fixate on the fixation target 220.

The method 800 includes receiving, at step 804, one or more images from the one or more cameras 144 having the eye to be treated in the field of view thereof. The images received at step 804 may be received in the form of one or more video feeds from the one or more cameras 144.

The method 800 includes locating, at step 806, the limbus of the eye 106 to be treated represented in the one or more images. Step 806 may be performed by registering one or more labeled reference image with respect to the one or more images, the labeled reference image including a label of the limbus. Step 806 may be performed using a machine learning model trained to perform the task, machine vision algorithm, or other approach. Step 806 may additionally or alternatively include identifying one or more other items of anatomy in the one or more images. For example, other items of anatomy may include the lens and the retina identified using images from an OCT.

The method 800 may include selecting, at step 808, an entry point relative to the limbus. For example, any point within a band of permitted offsets from the limbus, such as between 3 and 3.5 millimeters for an aphakic eye and between 3.5 and 4 millimeters for a phakic/pseudophakic eye. The angular position of the entry point about the optical axis of the eye 106 to be treated may be selected as a position that is not obscured by an eyelid of the patient. The entry point may be selected based on positions of other items of anatomy, such as the lens and retina from step 806, in order to avoid damage to the other items of anatomy or to deliver drugs to one or more other items of anatomy, such as to a sub-retinal space. In some embodiments, the controller 140 generates a three-dimensional model of the eye 108 and uses the model to precisely select the entry point and orientation of the needle in order to avoid damaging ocular tissue, such as the lens, retina, or other items of anatomy.

The method 800 may include actuating, at step 810, the staging assembly 204 such that the needle 208 of the needle assembly 206 is pointed at the entry point along the actuation direction of the extension actuator 214. The desired angle is as known in the art of intravitreal injections and may be selected such that upon insertion of the needle 208, the needle avoids contact with the lens and retina while placing medication near the retina or area of the retina to be treated. Note that in some applications, the needle will be relatively short (e.g., about 8 mm) such that the angle and depth are not critical for avoiding harm to ocular tissue. In other applications, the needle is used to provide a sub-retinal injection such that angle and depth of penetration are important. In some embodiments, if the range of motion of the staging assembly 204 is not sufficient to position the needle 208 pointed at the selected entry point, the method 800 may end or the user may be instructed how to adjust the patient's head 104 relative to the staging assembly 102 to make proper positioning possible.

Step 810 may be performed along with one or more additional iterations of some or all of steps 804, 806, 808 to account for movement of the eye 106 to be treated. Likewise, step 810 may include identifying a representation of the needle 208 in the one or more images received from the one or more cameras 144 and using the representation as feedback to guide positioning of the needle relative to the selected entry point.

The method 800 may include transmitting, at step 812, real time data to an observer, such as to the computing device 608 of an authenticated medical professional. The real time data may include images from the one or more cameras 144, such as by forwarding a video feed from the one or more cameras 144. The real time data may include a representation of the selected entry point from step 808 and a location and orientation of the needle, such as in the form of annotations to images from the one or more cameras 144. The real time data may include reports of successful application of anesthetic and disinfectant, which may include an amount of each applied. The real time data may include outputs of one or more interlock sensors 604 indicating whether the patient is properly positioned and components of the drug delivery device are locked in place and functioning correctly.

The method 800 may include performing one or more verifications prior to inserting, at step 818, the needle 208 into the eye 106 and administering an injection or drawing out tissue to perform a biopsy. In some embodiments, some or all of steps 802-812 may be repeated until the verifications are successful or the method 800 is ended by the patient or the observer. The verifications may include verifying, at step 814, that authorization was received from the observer and verifying, at step 816, that fixation of the eye 106 to be treated has been maintained. For example, step 816 may include verifying, using a video feed from the one or more cameras 144, that movement of the eye 106 to be treated is below a maximum threshold, e.g., less than 1 degree, 0.5 degrees, or 0.1 degrees. Step 816 may include verifying that fixation (e.g., movement less than the maximum threshold) was maintained for at least a minimum time period, e.g., from 1 to 3 seconds. Other verifications may include verifying the identity of the patient, such as by verifying that an iris or retina in one or more images from the one or more cameras 144 matches one or more reference images of an iris and/or retina or representation thereof accessed by the controller. In some embodiments, an explicit instruction can be received from the patient to verify that step 818 can be performed, such as in the form of pressing or releasing a button, a verbal command, or visible gesture detected by a camera coupled to the controller 140.

Administering the injection or performing the biopsy at step 818 may include activating the extension actuator 214 to drive the needle 208 into the eye 106 to be treated and activating a plunger actuator 504 or pump 526 to force fluid through the needle and into the eye 106. Step 818 may be performed simultaneously with one or more actions that may include verifying continued authorization by the observer. For example, an observer may continue to receive a video feed from the one or more cameras 144. The observer may hold a button throughout the procedure and release the button in the event that the observer believes that the injection should be aborted. In response to receiving notification of release of the button, the controller 140 may abort the injection. This approach to continued authorization is exemplary only and other approaches may be used, such as the observer pressing a button or interacting with another user interface element to invoke transmission of an instruction to the controller 140 to abort the injection by the controller 140. Step 818 may likewise be aborted in response to an input from the patient in the form of pressing or releasing a button, a verbal command, or visible gesture detected by a camera coupled to the controller 140.

Likewise, fixation may continue to be evaluated as described above with respect to step 816 and possibly by sensing strain on the needle. In the event that fixation is not maintained the injection may be aborted. The IOP within the eye 106 to be treated may be evaluated using outputs of the one or more IOP sensors 222. In the event that the IOP rises fastener than a prescribed rate or above a prescribed pressure, the injection may be aborted or the rate of drug delivery may be slowed. In some embodiments, the rate of injection of fluid is regulated based on feedback regarding IOP in order to maintain pressure within the eye 108 below a threshold or a pressure-vs-time curve, with time being measured from when fluid injection began.

In some embodiments, the staging assembly 204 may be activated during step 818 in order to at least partially compensate for movement of the eye 106 to be treated relative to the needle. For example, the staging assembly 204 may include one or more strain sensors sensing strain on the needle 208 in one or more dimensions. The controller 140 may activate one or more actuators of the staging assembly 204 to reduce the amount of strain sensed by the strain sensors. Step 818 may be aborted in response to movement of the eye 106 to be treated exceeding the range of motion and/or speed of movement required for the staging assembly 204 to compensate for the movement of the eye 106.

Aborting the injection or the biopsy may include causing the actuator extension actuator 214 to withdraw the needle 208 of the needle assembly 206 from the eye 106 to be treated to a safe distance from the eye 106 to be treated. Once aborted, the controller 140 may require repetition of the method 700 and 800. Alternatively, once aborted, the controller 140 may disable further injections or extraction of biopsy samples and require the patient to visit a medical professional.

Step 818 may include monitoring the amount of drug delivered, e.g., amount by which a plunger of a syringe was depressed, or amount of pumping performed by the pump 526. Accordingly, an amount of drug that remains to be administered may be determined by the controller 140 and provided to an observer or used by the controller 140 to control the amount of drug delivered in a subsequent iteration of the method 800.

Once the methods 700 and 800 are performed for one eye 106 to be treated, the methods 700 and 800 may be repeated for the patient's other eye 106. Alternatively, the docking assembly 102 may include two staging assemblies 204. The methods 700 and 800 may be performed for each eye 106 of the patient in series, in parallel, or in an interleaved manner. For example, administration of disinfectant and anesthetic may be performed for both eyes 106 in parallel whereas fixation and injection or biopsy extraction (e.g., steps 814-818) may be performed in series.

The methods 700 and 800 are exemplary only and may be modified to perform additional steps or ophthalmic treatments. For example, although a drug to be delivered, an anesthetic, and a disinfectant are mentioned above, other fluids may also be used to treat the eye 106 either before or after an injection. For example, some or all of a cooling spray (e.g., saline), anti-inflammation cream or spray, anti-bleeding solution may also be loaded into the needle assembly 206 and applied using the needle assembly 206. In some embodiments, the staging assembly 204 may include one or more actuators that are activated by the controller to press a pad (e.g., cotton or other absorbent material) against an injection site following injection in order to reduce bleeding. Likewise, an actuated pad incorporated into the staging assembly 204 may be pressed against the eye 106 during injection to resist movement of the eye.

### ADDITIONAL CONSIDERATIONS

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A delivery device comprising:
a conveyor;
one or more imaging devices configured to have an eye of a patient in a field of view thereof;
a needle assembly comprising a needle;
a staging assembly mounted to the conveyor, the conveyor configured to move the staging assembly in three-dimensional space, the staging assembly including one or more actuators and configured to position the needle assembly relative to the eye of the patient; and
a controller coupled to the conveyor, the one or more imaging devices, and the staging assembly, the controller configured to:
receive one or more images from the one or more imaging devices;
detect a location of anatomy of the eye of the patient in the one or more images; and
activate the one or more actuators to drive the needle into a placement location on the eye of the patient according to the location of the anatomy.

2. The delivery device of claim 1, further comprising one or more clamping actuators configured to restrict movement of a head of the patient.

3. The delivery device of claim 2, wherein the one or more clamping actuators are mounted to the conveyor, the conveyor configured to move the one or more clamping actuators in the three-dimensional space.

4. The delivery device of claim 2, wherein the one or more clamping actuators are mounted to a support to which the conveyor is mounted.

5. The delivery device of claim 1, wherein the conveyor has at least five degrees of freedom.

6. The delivery device of claim 1, wherein the conveyor is a robotic arm.

7. The delivery device of claim 1, wherein the needle assembly is configured to at least one of force fluid through the needle into the eye of the patient or draw a tissue sample out of the eye of the patient.

8. The delivery device of claim 1, further comprising a fixation target that is fixed relative to the staging assembly, the controller being further configured to activate the one or more actuators to drive the needle into the placement location in response to verifying fixation of the eye of the patient on the fixation target.

9. The delivery device of claim 1, wherein the controller is further configured to activate the one or more actuators to drive the needle into the placement location in response to an authorization received from a remote observer over a network.

10. The delivery device of claim 1, wherein the one or more actuators include two arcuate actuators and a linear actuator.

11. A method for drug delivery comprising:
activating, by a controller, a conveyor to transport a staging assembly toward an eye of a patient, the staging assembly having an needle assembly mounted thereto;
receiving, by the controller, one or more images from one or more imaging devices mounted to the staging assembly;
detecting a location of anatomy of an eye of the patient in the one or more images;
activating, by the controller, one or more actuators of the staging assembly to align the needle assembly relative to the location of the anatomy; and
activating, by the controller, the one or more actuators to drive a needle mounted to the needle assembly into a placement location on the eye of the patient according to the location of the anatomy.

12. The method of claim 11, further comprising activating, by the controller, one or more clamping actuators, to restrict movement of a head of the patient.

13. The method of claim 12, wherein the one or more clamping actuators are mounted to the conveyor, the conveyor configured to move the one or more clamping actuators in three-dimensional space.

14. The method of claim 12, wherein the one or more clamping actuators are mounted to a support to which the conveyor is mounted.

15. The method of claim 11, wherein the conveyor has at least five degrees of freedom.

16. The method of claim 11, wherein the conveyor is a robotic arm.

17. The method of claim 11, further comprising activating, by the controller, the needle assembly to at least one of (a) force fluid through the needle into the eye of the patient or (b) draw tissue out of the eye of the patient.

18. The method of claim 11, further comprising:
activating, by the controller, a fixation target that is fixed relative to the staging assembly;
verifying, by the controller, fixation of the eye of the patient on the fixation target; and
activating, by the controller, the one or more actuators to drive the needle into the placement location in response to verifying fixation of the eye of the patient on the fixation target.

19. The method of claim 11, further comprising:
receiving, by the controller, authorization from an observer; and
activating, by the controller, the one or more actuators to drive the needle into the placement location in response to receiving the authorization.

20. The method of claim 11, wherein the one or more actuators comprise two arcuate actuators and a linear actuator.
